SEQUENCE LISTING (13 sequences)

SEQ ID NO: 1 - DNA, 117 bp, Hordeum vulgare
ggtggcaaaaagtctagcagtagtagttccctgatgtacgaagctcccctcggctacagcattgaagacgttcgaccagctggaggcgcaaagaagttttctgctgcataytcaaac

SEQ ID NO: 2 - DNA, 109 bp, Hordeum vulgare
tgcgcgaagaagccatcctgatatcgtttttggctttccccttcccgtagtttaggatttttaactgctgcaattttattctgactcttttctcccaccaatctctctg

SEQ ID NO: 3 - DNA, 23 bp, Artificial Sequence
  Oligonucleotide
agaagttttctgctgcataytca

SEQ ID NO: 4 - DNA, 24 bp, Artificial Sequence
  Oligonucleotide
tggctttccccttcccgtagttta

SEQ ID NO: 5 - DNA, 21 bp, Artificial Sequence
  Oligonucleotide
taaactacgggaaggggaaag

SEQ ID NO: 6 - DNA, 19 bp, Artificial Sequence
  Oligonucleotide
agaagttttctgctgcata

SEQ ID NO: 7 - DNA, 24 bp, Artificial Sequence
  Oligonucleotide
taaactacgggaaggggaaagcca

SEQ ID NO: 8 - DNA, 23 bp, Artificial Sequence
  Oligonucleotide
tgartatgcagcagaaaacttct

SEQ ID NO: 9 - DNA, 23 bp, Artificial Sequence
  Oligonucleotide
tcttcaaaagacgacgtatyagt

SEQ ID NO: 10 - DNA, 24 bp, Artificial Sequence
  Oligonucleotide
accgaaaggggaagggcatcaaat

SEQ ID NO: 11 - DNA, 19 bp, Artificial Sequence
  Oligonucleotide
atacgtcgtcttttgaaga

SEQ ID NO: 12 - DNA, 23 bp, Artificial Sequence
  Oligonucleotide
tcttcaaaagacgacgtatytca

SEQ ID NO: 13 - DNA, 20 bp, Artificial Sequence
  Oligonucleotide
ratacgtcgtcttttgaaga

(19) United States
(12) Patent Application Publication
(10) Pub. No.: US 2006/0127932 A1
(43) Pub. Date: Jun. 15, 2006

(54) Method for the SNP analysis on biochips having oligonucleotide areas

(76) Inventors: Dirk Fischer, Gatersleben (DE); Jorg Geistlinger, Gatersleben (DE)

Correspondence Address: KNOBBE MARTENS OLSON & BEAR LLP, 2040 MAIN STREET, FOURTEENTH FLOOR, IRVINE, CA, 92614, US

(21) Appl. No.: 11/293,048

(22) Filed: Dec. 2, 2005

Related U.S. Application Data
(63) Continuation of application No. PCT/EP04/06002, filed on Jun. 3, 2004.

(30) Foreign Application Priority Data
Jun. 3, 2003 (DE) 103 25 098.0

(51) Int. Cl. C12Q 1/68; C12M 1/34

(52) U.S. Cl. 435/6; 435/287.2

(57) ABSTRACT

The invention relates to a method for the multiparallel detection of nucleotide polymorphisms on a polydimensional array. The invention also relates to a method for detecting many individual nucleotide polymorphisms, during which the nucleotide polymorphisms of multiple individuals can be multiparallelly detected on the array. According to the invention, the hybridization of a probe molecule with a sample molecule ensues in a hybridization field on the array that is separated from surrounding hybridization fields.

The present application is a continuation of and claims priority under 35 U.S.C. §120 to PCT Application Serial No. PCT/EP2004/006002, filed Jun. 3, 2004 by Fisher et al., entitled “METHOD FOR CONDUCTING SINGLE NUCLEOTIDE POLYMORPHISMS (SNP)-ANALYSIS ON BIOCHIPS HAVING OLIGONUCLEOTIDE AREAS,” and which claims priority to German Patent Application No. 103 25 098.0, filed Jun. 3, 2003, both of which are herein incorporated by reference in their entirety.

### BACKGROUND OF THE INVENTION

1. Field of the Invention

The present invention relates to a method for the multi-parallel detection of nucleotide polymorphisms on a polydimensional array. Furthermore, the invention relates to a method for the detection of a multitude of single nucleotide polymorphisms, wherein the nucleotide polymorphisms of multiple individuals can be detected on the array in a multi-parallel manner. Within the scope of the method according to the invention, hybridization of a probe molecule with a sample molecule occurs in a hybridization field on the array which is separated from surrounding hybridization fields.

2. Description of the Related Art

Naturally occurring DNA variations among individuals have been examined for several years in the fields of pharmacogenomics and pharmacogenetics and generally for genotyping human, animal, microbial, fungal and herbal individuals. One type of DNA sequence variations, distinguishing the DNA of one individual from that of another individual, are the so-called “single nucleotide polymorphisms” (SNPs) which also exist among individuals of the same species.

An SNP is an exchange of a single DNA base or an insertion/deletion of single bases at a specific position within a genomic region such as a gene. Several individuals in a population may e.g. have the base adenine, while other individuals have the base cytosine at the same position within a gene. More than 5 million of such SNPs, whose allele bearing the less frequent nucleotide appears in more than 10% of the examined individuals, are expected to exist in the human genome. Of these, about 90,000 SNPs are supposed to be located in protein encoding regions, thus being of special interest with regard to medical and pharmacological issues. The examination of several SNPs in an individual offers the possibility to establish a genetic fingerprint of the individual under concern. These differences can be examined and analyzed by means of DNA microarrays specifically designed for SNP analysis. SNP analysis is generally useful to clarify issues of molecular genetics. The result of an SNP analysis may e.g. reveal valuable information about an individual's predisposition for a certain disease. In this manner it is possible to estimate the patient's reaction to a specific class of active agents before the medication of the patient.

However, SNP analysis, often also referred to as genotyping, is not only of special interest for medical genetics and pharmacogenetics; the determination of the presence or absence of a certain SNP and therefore of a certain property in an individual is generally useful for the characterization of individuals, may they be of human, animal, herbal or microbial nature. Therefore, SNP genotyping has been an inherent part of modern plant breeding for several years now.

Since that time, SNP analysis is applied routinely, especially in the area of biomedicine, and is offered as a service by companies oriented accordingly. Furthermore, suitable reaction kits for SNP analysis are already commercially available from different suppliers, but not for arrays.

Two methods for the detection of point mutations are predominantly applied on a DNA chip or microarray, the use of which for SNP analysis is offered as a service by different companies: allele specific hybridization on the one hand and primer extension on the other hand. These detection methods are based on oligonucleotides which are arranged on the chip in the form of a so-called array, i.e. a predetermined arrangement, in order to detect either via hybridization or via hybridization with subsequent DNA polymerase dependent primer extension of the arranged oligonucleotides. For both techniques, the probe, i.e. the respective oligonucleotide, is arranged and fixed at a specific position on the chip, while the nucleic acid molecules of the sample to be examined are present in the form of a hybridization solution. This solution is brought into contact and incubated with the chip, allowing the DNA molecules of the sample, which are present in the solution, to find their appropriate hybridization partner, i.e. the oligonucleotide probe matching the respective molecule, on the surface of the biochip and to hybridize with it.

The terms “DNA chip”, “biochip” and “(micro) array” are often used as synonyms, which is also the case in this application. In a stricter sense, “microarray” only means that molecules are arranged with high density at defined positions within an arrangement (array) or pattern. In fact, microarrays can exhibit up to several hundred thousand positions (often referred to as “spots”) on a carrier or a matrix. The chip is the actual array substrate, i.e. the carrier for one or, more commonly, several microarrays. Object slides or other glass substrates and wafers are used, inter alia, as carriers.

In genotyping, i.e. the detection of the presence or absence of an SNP using the currently common microarray-techniques, numerous DNA molecules or loci of a single individual normally have to find their different hybridization partners. Obviously, several problems result therefrom, especially with increasing numbers of loci to be examined. Sample molecules frequently do not find their matching probes, especially when the experiment is complex and the solution contains a large number of sample molecules, and a large number of immobilized oligonucleotide probes, e.g. 1,000 or more, are located on the chip surface. In such cases the hybridization result is often poor, and many sample molecules never find their probe partner, consequently leading to missing signals and wrong results. Furthermore, the sample molecules or the loci to be examined have to derive from one single individual, since in the case of using two or more individuals or their samples, cross-hybridization of the homologous sample molecules with the same probe would occur automatically, and the signals would therefore be undistinguishable and unrelatable.

Similar problems occur not only in the parallel detection of several SNPs of various individuals, but also in all microarray based methods which are based on a multitude of parallel hybridization events between probes and targets. This is especially the case in the detection of similar sequences.

A typical example for such methods is the use of microarrays for the detection of microorganisms in samples for biomedical diagnostics. This method exploits the fact that the genes for ribosomal RNA (rRNA) are ubiquitous and contain sequence regions which are characteristic for the respective species. These species-characteristic sequences are applied on a microarray as single-stranded DNA oligonucleotides. The target DNA molecules to be examined are first isolated from the sample to be examined and coupled with fluorescence markers. The labeled target DNA molecules are then incubated in a solution with the probes applied to the microarray, unspecific interactions are removed by means of appropriate washing steps, and specific interactions are detected via fluorescence-optical analyses. In this manner, it is possible to simultaneously detect e.g. several microorganisms in one sample with one single test. In this test procedure the number of detectable microorganisms theoretically only depends from the number of specific probes which have been applied to the microarray. Yet, since quite often microorganisms with very similar rRNA sequences are to be detected in parallel, the problems described for SNP analysis, based on competition, cross-hybridization etc., arise here too.

The technical possibilities to compensate for the mentioned disadvantages by using different lasers and fluorochromes are very limited. This results in the fact that, e.g. for SNP analysis for genotyping each single individual, a separate chip has to be produced and applied, as described e.g. in Science (2002), 295, pages 160-172, where a current overview of DNA chip technologies and microarray technologies can be found, see especially page 172, 3^{rd} column. The manufacturing of individual microarrays for each individual to be examined is that time-consuming and cost-intensive that this procedure, e.g. in a trial with 5,000 test persons, which would cost almost 5 million Euro in view of the necessity of 5,000 SNP chips only for the hybridization detection step, is hardly applicable.

Although SNPs and genotyping of patients are of central importance especially in medical genetics, no practicable attempts, which overcome the disadvantages of the prior art and allow multi-parallel analysis of numerous loci of many individuals simultaneously, have been developed until now.

### BRIEF DESCRIPTION OF THE DRAWINGS

FIG. 1: Hordeum vulgare L. (barley) EST locus, SEQ ID NOS. 1 and 2, from the NCBI Genbank database (http://www.ncbi.nlm.nih.gov/data base entry: gi9410596, bold, underlined: primer site, bold, italics, underlined: complementary PTO primer site, Y: SNP)

FIG. 2: Genomic PCR of the SNP containing fragment (size: 174 bp, forward primer: gray on black background, PTO reverse primer: gray background; nucleotides linked via phosphothioate bindings: white, bold, black background), SEQ ID NOS. 3-8.

FIG. 3: Annealing (PTO protected fragment at the extension primers fixed to the area) in the hybridization field of the area and before the extension: formation of a hybrid molecule between the extension primer (oligonucleotide in box) with amino C₁₂-modification on the one hand and the complementary PTO protected single strand on the other hand (SNP: bold, larger, gray background), SEQ ID NOS. 9-11.

FIG. 4: Extension reaction on the chip

Enzymatic attachment of exactly one fluorescently labeled dideoxynucleotide or other terminator complementary to SNP (white, larger, gray background: ddRTP is ddGTP or ddATP) at the hybridized extension primer, SEQ ID NOS. 12 and 13.

FIG. 5: Process steps to be performed with the object slide (Note: the drawing is original scale, with the exception of the vertical extent of area (a) in FIG. 5/IV and V, which is only depicted for better visualization:
I. Plane coating of the object slide (s) with oligonucleotide probes in hybridization areas (a),
II. Application of the separation matrix (g) on the object slide (s) coated with probe molecules in hybridization areas (a) and provided with a bar code (b),
III. Application of the sample molecules (p) in hybridization fields (f) within the hybridization areas (a) separated from each other by the separating matrix (g),
IV. Fixing the lid (d) to perform the extension reaction in the hybridization areas (a) separated from each other by the separation matrix (g) on the object slide (s),
V. Sealing the object slide against the lid with a silicone rubber sealing (o) and analysis of the sample spots (p) after primer extension,
VI. Alternative embodiment for the hybridization of 16 sample molecules in hybridization fields (f) with probe molecules immobilized in 192 hybridization areas,
VII. Alternative embodiment for the hybridization of 4800 sample molecules (p) in hybridization fields (f) with probe molecules of the same sequence immobilized in a hybridization area (a).

### DETAILED DESCRIPTION OF THE PREFERRED EMBODIMENT

It is therefore an object of the invention to enable parallel analysis of several nucleic acids via hybridization simultaneously and comparably on an array, wherein sources of error (e.g. cross-hybridization) should be eliminated as far as possible and no high demands should be placed on analytical parameters such as sample concentration.

It is also an object of the invention to enable the analysis of multiple diagnostic mutations or SNPs simultaneously and comparable at many loci on an array. If possible, no high demands should be placed on the different analytical parameters, like identical hybridization temperature or DNA sample concentration on the array. A detection method as simple and reliable as possible, with as little sources of error as possible, should rather be provided. For this reason, a complicated cyclic temperature regime, as it is required e.g. for PCR, should also be avoided.

These and other objects of the invention, as they are resulting from the description, are achieved by the subject-matter of the independent claim. Preferred embodiments of the invention are defined in the subclaims.

The objects mentioned are achieved by means of the method according to the invention for simultaneous genotyping of multiple individuals and multiple loci on one and the same carrier (DNA chip or array).

The prior art techniques for microarray based detection of nucleic acids generally require that sample molecules or target molecules are pooled in the hybridization solution, and that the hybridization solution is brought into contact with the chip, on which the oligonucleotide probes are arranged, which leads to the requirement that the different sample molecules have to find their hybridization partner among a multitude of probes, causing problems especially with high numbers of sample molecules.

In contrast to the above, the invention is based on the principle that only one sample molecule (in the meaning of one defined nucleic acid sequence) hybridizes with one complementary probe molecule (in the meaning of one defined nucleic acid sequence) immobilized on the chip, in discrete hybridization fields on the biochip. Therefore, a locally targeted hybridization occurs within the scope of the method according to the invention, in which unwanted hybridization reactions are excluded due to the spatial distance of the individual hybridization fields and hybridization areas and due to optional drying of the generated hybrids.

For the detection of SNPs, the prior art techniques also require that the sample molecules of a single individual are pooled in the hybridization solution and that the hybridization solution is brought into contact with the chip on which the oligonucleotide probes are arranged, which leads to the requirement that different sample molecules of an individual have to find their hybridization partner among a multitude of probes, causing problems especially with increasing numbers of analyzed loci. The invention, in contrast, is based on the fact that no pool of samples is submitted to hybridization with the respective probe, that every single sample molecule (in the meaning of a distinct nucleic acid sequence) is hybridized exclusively with the complementary probe molecule in a separate hybridization field. In this manner, hybridizations with other sample molecules (in the meaning of a distinct nucleic acid sequence) (cross-hybridization) are prevented. In the scope of the method according to the invention, targeted hybridization occurs between a sample molecule and the complementary probe molecule, without allowing the different sample molecules to mix.

Within the scope of the present invention for SNP detection, the formation of the hybrid of probe molecule and sample molecule takes place individually for each locus to be examined and each individual to be examined. Hybridization occurs in spatially distant hybridization fields on the chip. With the position of the oligonucleotide probe having been fixed in advance, it is possible to securely correlate the hybrids formed from probe and sample.

The formed hybrids can be stabilized by the addition of, e.g., spermidine or polyethylene glycol, or preserved under alcohol. The stabilization of the hybrid structure, i.e. the cohesion of the single strands in the double stranded, is supported by drying, i.e. by the reduction of the volume.

In a preferred embodiment the hybrids of oligonucleotide probe and sample molecule are dried for further protection from mixing of the different sample molecules. These dried hybrids are rehydrated during the detection reaction.

In an especially preferred embodiment the hybridization areas are additionally separated from each other by applying a separating matrix made of plastic, which can be removed again before the detection reaction.

It is obvious to the person skilled in the art that the method according to the invention for SNP detection can in principal be extended to other microarray based detection procedures. According to the invention, e.g., for the detection of microorganisms in a sample, PCR amplified target molecules can also undergo hybridization with rDNA probe, specific for a microorganism, on the chip, without mixing of the target molecules. In this manner, competing reactions of different probes for targets can easily be eliminated and cross-hybridizations can be avoided. Since only one probe is available for hybridization, hybridization can be performed with higher efficiency, leading to improved signal-to-noise ratios in the analysis. Furthermore, problems arising from simultaneous diffusion of numerous targets to a multitude of probes, which appear in the analysis of prior art techniques, are avoided.

Other applications can e.g. be the analysis of transcriptional activity of an organism or e.g. of a cell. In this case, firstly, the mRNA, e.g. the expressed genetic information, is quantitatively transformed into the corresponding cDNA via reverse transcription. After amplification of these cDNAs, in a method according to the invention, hybridization of an amplified cDNA with a probe in a spatially defined hybridization field and analysis of the hybridization can be performed in order to draw conclusions about the genetic activity of the respective cell, etc.

Alternatively, single stranded mRNA, e.g. from a highly expressed or strongly induced gene, can be used directly as sample molecule submitted to hybridization with the probe molecules, in order to investigate the expression of these genes. The application of the procedure according to this invention for analyses on the RNA level is therefore explicitly intended.

Further applications of the method according to the invention, such as analyses of mutations, analyses of recombinations, genealogical analyses and segregation analyses are well known to the person skilled in the art.

The invention therefore relates to a method for the detection of nucleic acids, comprising the following steps in the order given:
a) plane arrangement and immobilization of one or more single-stranded nucleic acid molecules (probe molecules) in hybridization areas having a minimum size of 0.5 mm² on a carrier,
b) mixing and hybridization of a specific probe molecule in a spatially defined hybridization field within the hybridization area with a second single-stranded nucleic acid molecule (sample molecule), whose sequence is at least partly complementary to that of the probe molecule, therefore allowing the hybridization of the two nucleic acid molecules at least over the range of the complementary sequence, wherein sample molecules of the same locus, but of different origin, can hybridize with the same probe molecule in different hybridization fields within one hybridization area,
c) detection of the hybridization with a suitable detection method.

In one preferred embodiment the hybrids forming in a hybridization field are dried, while or before probe molecule and sample molecule are brought into contact with each other in the neighboring hybridization field.

In one especially preferred embodiment the hybrids forming in a hybridization field are dried, while or before probe molecule and sample molecule are brought into contact with each other in the neighboring hybridization field, wherein a separating matrix is applied between the discrete hybridization areas, separating the hybridization areas from each other.

The method of invention is suitable for the parallel detection of e.g. several sample molecules in a mixture, for the parallel detection of several organisms, preferably microorganisms in a sample, for the analysis of gene activity of multiple genes of an organism or e.g. a cell, and also for the detection of the expression of a gene.

In one preferred embodiment the invention relates to a method for the detection of a nucleotide polymorphism (SNP), comprising the following steps in the order given:
a) plane arrangement and immobilization of one or more single-stranded nucleic acid molecules (probe molecules), whose sequence represents a specific locus in the genome of an individual, in hybridization areas having a minimum size of 0.5 mm² on a carrier,
b) mixing and hybridization of a specific probe molecule in a spatially defined hybridization field within the hybridization area with a second single-stranded nucleic acid molecule (sample molecule), which contains the SNP to be detected and whose sequence is at least partly complementary to that of the probe molecule, therefore allowing the hybridization of the two nucleic acid molecules at least over the range of the complementary sequence, wherein sample molecules of the same locus, but of different origin, can hybridize with the same probe molecule in different hybridization fields within one hybridization area,
c) detection of the SNP via analysis of the hybridization reaction using a suitable detection method.

In one preferred embodiment the hybrids forming in a hybridization field are dried, while or before probe molecule and sample molecule are brought into contact with each other in the neighboring hybridization field.

In one especially preferred embodiment the hybrids forming in a hybridization field are dried, while or before probe molecule and sample molecule are brought into contact with each other in the neighboring hybridization field, wherein a separating matrix is applied between the discrete hybridization areas, separating the hybridization areas from each other.

The method of the invention is therefore suitable for the detection or genotyping of the presence, absence or identity of a single nucleotide polymorphism at a defined position within the genome of an individual. Preferably, several to numerous individuals are analyzed simultaneously on one common carrier with respect to a specific nucleotide variation. Especially preferably is the simultaneous analysis of a large number of individuals with respect to only a few nucleotide variations.

The method of the invention is also suitable for the detection or genotyping of the presence, absence, or identity of insertions or deletions of one or more bases at a certain position within the genome of an individual.

This invention furthermore relates to a method for the multi-parallel SNP analysis of several individuals and several loci within the genome of an individual, comprising the following steps in the order given:
a) plane arrangement and immobilization of one or more single-stranded nucleic acid molecules (probe molecules), whose sequence represents a specific locus in the genome of an individual, in hybridization areas having a minimum size of 0.5 mm² on a carrier,
b) mixing and hybridization of a specific probe molecule in a spatially defined hybridization field within the hybridization area with a second single-stranded nucleic acid molecule (sample molecule), which contains the SNP to be detected and whose sequence is at least partly complementary to that of the probe molecule, therefore allowing the hybridization of the two nucleic acid molecules at least over the range of the complementary sequence, wherein sample molecules of the same locus, but of different origin, can hybridize with the same probe molecule in different hybridization fields within one hybridization area,
c) enzymatic template dependent extension of the probe molecule, which acts as a primer within the hybrid, via incorporation of a nucleotide or nucleotide analogue resulting in chain termination,
   wherein probe molecules representing different loci and sample molecules of several individuals are used for parallel analysis.

In one preferred embodiment the hybrids forming in a hybridization field are dried, while or before probe molecule and sample molecule are brought into contact with each other in the neighboring hybridization field.

In one especially preferred embodiment the hybrids forming in a hybridization field are dried, while or before probe molecule and sample molecule are brought into contact with each other in the neighboring hybridization field, wherein a separating matrix is applied between the discrete hybridization areas, separating the hybridization areas from each other.

In a further embodiment of the invention, in each hybridization area, only one sample molecule undergoes hybridization with the probe molecule present in the area, wherein each area can be spatially separated from the other areas by a separating matrix.

According to the invention, probe molecules representing different loci of different individuals can be used for parallel analysis.

Different embodiments of the method according to the invention for the use of arrays for multiparallel SNP analysis of multiple individuals and multiple loci in the genome of an individual are defined in the subclaims.

According to the invention, the term “locus” means a genomic section which includes the genetic polymorphism to be examined, such as the SNP, the insertion or the deletion.

Within the scope of the present invention, the term “immobilization” means the procedure in which a molecule is applied to a solid carrier or a layer or surface stabilized thereon by covalent or non-covalent interactions in a manner preventing free movement of the molecule on the carrier or diffusion from the carrier back to solution. Within the scope of the present invention, the immobilization of the probe molecules in the areas results in a plane coating with the oligonucleotide probe.

Within the scope of the present invention, the terms “carrier”, “matrix” or “substrate” are used to describe devices on which molecules can be applied via covalent or non-covalent interactions. Suitable carriers or substrates for nucleic acid molecules can e.g. be object slides or other materials consisting of glass, plastics, ceramics or metal, which can be coated in a planar, two-dimensional, three-dimensional or spherical manner, as well as wafers. Three- and four-dimensional coatings for carriers are commercially available, e.g. under the designations CodeLink Bioarray (Amersham Pharmacia) or MGX™ 4D-Array (Metrigenix)

In a preferred embodiment, the different hybridization areas are separated from each other by a separating matrix made from chemically inert material, in contrast to carriers with wells or other cavities, as they are known in the prior art.

Within the scope of the present invention, the term “hybridization area” is used to describe the region of the chip in which an oligonucleotide probe of defined sequence is arranged in a plane manner and immobilized. A chip preferably contains at most 192 areas, especially preferably at most 96 areas, particularly preferably at most 48 areas and most preferably at most twelve areas. The surface of the areas is at least 0.5 mm², preferably at least 4 mm², especially preferably at least 16 mm² and most preferably at least 256 mm². Multiple hybridizations can take place in spatially separated hybridization fields within these areas.

Within the scope of the present invention, the term “hybridization field” is used to describe the region within the hybridization area in which a sample molecule having a defined nucleic acid sequence originating from a certain individual undergoes hybridization wit a probe molecule, whose nucleic acid sequence is at least partly complementary to that of the sample molecule. The distance between the hybridization fields is at least 1.5 to 2 mm in the case of manual application of the sample molecule, and in the case of automatic application the distance can be 0.1 to 1 mm.

If the localization on the carrier, the matrix or substrate is carried out in a defined spatial arrangement, this arrangement is often called “array”. A specific position on the array, in the present case the hybridization field, is commonly also referred to as spot. For the person skilled in the art, it is obvious that the terms “array”, “substrate”, “matrix”, etc. are often used as synonyms in the prior art and can not be strictly distinguished by definitions.

The term “chip”, “DNA chip”, “biochip” or “gene chip” usually refers to the arrangement of a molecule forming an array on a carrier.

Within the scope of the present invention, the term “separating matrix” refers to a grid, separating the distinct hybridization areas on the chip from each other. The material used for the separating matrix may be any chemically inert material that effectively prevents the passage of a liquid and ensures reliable sealing of the separate areas against the carrier. Additionally the material should stick reversibly to the carrier, be tear resistant, and it should be possible to remove it after hybridization preferably in total and without residue. Chemically inert polymers and plastics are preferred. The especially preferred material for the separating matrix is silicone rubber. The width of the separating matrix is preferably 0.2 to 5 mm, especially preferably 0.5 to 3 mm and most preferably 0.8 to 1 mm. The height of the separating matrix should be preferably 0.2 to 2 mm, especially preferably 0.8 to 1.8 mm and most preferably 1.2 to 1.6 mm. The separating matrix can be removed after hybridization in order to allow a collective detection reaction for the whole chip.

The detection of an SNP in a sample molecule having hybridized with a probe and being immobilized on a carrier can be performed by various detection methods well known to the person skilled in the art. The most established and most commonly used method for the detection of SNPs in sample/probe hybrids is the so-called primer extension.

Primer extension, i.e. the extension of the probe molecule acting as a primer in a template dependent manner, wherein the sample molecule is the template, can be performed in a conventional manner, as described e.g. in EP 0 648 280 B1, EP 0 705 349 B1 and WO 98/59066 A1. It is herewith explicitly referred to the disclosure of primer extension contained in these documents.

For the primer extension which has become the common method for SNP analysis by now, primer molecules containing a polynucleotide sequence complementary to one or more nucleotide sequences of a genomic DNA segment of an individual, wherein the genomic segment is directly located 3′-distal to an SNP, Y, are elongated via template dependent extension of the nucleic acid primer molecule by a single nucleotide or nucleotide analogue R, which is complementary to the nucleotide Y of the SNP allele. If the template dependent extension of the primer molecule, which is, in technical terms, often called interrogation primer, takes place in the presence of one or more dideoxy nucleoside triphosphate derivatives or analogues, selected from the group consisting of ddATP, ddTTP, ddCTP, and ddGTP, or other base analogues leading to chain termination, but in the absence of dATP, dTTP, dCTP and dGTP, the dideoxynucleotide triphosphate derivative, which is not further extensible, can be detected in the position of the SNP, thereby allowing the detection of the SNP itself in a conventional manner. As mentioned above, the primer extension step allowing direct SNP detection has often been described in the prior art and can be performed by an person skilled in the art by means of commercially available reagents and reaction kits.

In general, the oligonucleotide applied as probe molecule for the formation of the hybrid made of probe molecule and sample molecule, also acts as primer, which is extended via a polymerase in a template dependent manner by the nucleotide under concern, which corresponds to the SNP allele.

Other detection methods for the detection of SNPs in the hybrid known to the person skilled in the art are e.g. allele specific primer extension (see below), allele specific hybridization (see below) and mass spectrometry.

The probe molecule/sample molecule hybrids formed on the chip according to the invention are not denatured at any time point before the extension, but rather provide the direct substrate for the polymerase which attaches a detectable, generally labeled nucleotide to the probe molecule under concern. Since the sample molecules to be examined hybridize with the corresponding probe molecules individually and separated from each other, any form of cross-hybridization is prevented, in contrast to prior art methods, where the sample molecules are pooled in a hybridization solution. This offers for the first time the possibility to analyze different hybrids of probe molecule and sample molecule representing different positions within a genome and therefore different loci, of different individuals and even of different species or populations in parallel on a single chip. Due to the present invention, multiple loci of multiple individuals can for the first time reliably be examined and analyzed in parallel on a single chip.

While the simultaneous and comparable analysis of multiple diagnostic mutations or SNPs at multiple loci on one array has so far been possible only by serial methods, either investigating discrete samples, i.e. one individual and one SNP per run, or by restricted parallel methods, i.e. investigating one individual with multiple loci in one run or one reaction space or hybridization space, now for the first time multiple individuals can be analyzed in parallel with respect to different loci, efficiently, cost-effectively and reproducibly in one experiment on a single chip. This is possible because in one preferred embodiment of the method according to the invention, each locus to be analyzed of each individual is amplified separately via PCR and undergoes hybridization with a probe characteristic for each locus and separated from other probes and samples in different hybridization fields within the areas on the chip.

Furthermore, the analysis can be carried out by the user himself, if ready-made chips, carrying the immobilized probe molecules in the hybridization areas and if necessary a removable separating matrix, are submitted to hybridization with previously amplified sample molecules by the user himself, thus detecting the polymorphism. This becomes possible by the spatially separated hybridization fields, allowing the sample molecules to be applied to the chip with a manual pipette or a pipette robot, preventing mixing of the molecules and therefore preventing cross-hybridization. The user therewith is enabled to perform the analysis of large numbers of individuals himself in a cost-effective, time-saving and reproducible manner. It thus becomes possible e.g. to produce chips with known SNPs related to the predisposition for certain diseases, enabling diagnostic laboratories to examine multiple individuals with regard to their predisposition to different diseases by simple means using one chip. The same applies of course for modem plant breeding, where multiple plant individuals are to be examined e.g. with regard to a yield raising SNP.

The method according to the invention also allows a certain degree of automation since the probe molecules can be applied to the hybridization areas on the matrix by means of a microarray- or pipette robot, and the sample molecules do not necessarily have to be applied with a manual pipette, but can also be applied to every hybridization field by means of a pipette- or microarray robot. Up to date, such automation involved the loss of polydimensional processing.

Furthermore the method according to the invention includes the manufacture of kits containing a prefabricated chip coated with one or more probe molecules in hybridization areas and optionally having a removable separation matrix. Additionally this kit may contain primer for PCR amplification of the sample molecules as well as reagents for the generation of single strands from the PCR products. Furthermore the kit may also contain the reagents for template dependent extension of the probe molecule. In a preferred embodiment of the invention the kit contains a pre-fabricated chip, coated with one ore more probe molecules in hybridization areas and having a removable separating matrix, primer for PCR amplification of the sample molecules, as well as reagents for the generation of single strands from the PCR products, and reagents for the template dependent extension of the probe molecule.

The method according to the invention for the detection of SNPs or for the production of multi-parallel SNP arrays is described in detail below, with regard to the individual steps and the hybridization partners.

The sample nucleic acid, herein also referred to as target nucleic acid, which is believed to contain the variable nucleotide residue, i.e. the SNP, and which is therefore to be analyzed, may be an nucleic acid (DNA or RNA) of human, animal, plant, fungal or microbial origin. The sample nucleic acid may be isolated from biological samples via conventional methods of nucleic acid purification, or may be present in an unpurified form within the biological sample.

Individual DNA fragments, which are believed to carry a variable nucleotide residue, i.e. an SNP, are amplified from isolated (purified) DNA or RNA or directly from non-purified biological samples (e.g. cellular sap or squeezed preparation, so-called crude extracts) using the respective PCR primers. This is possible, if the sample contains so-called recoverable DNA material. It is known to the person skilled in the art that RNA can be transformed by reverse transcription into cDNA, which can then be used as a template e.g. in a PCR.

The methods for enzymatic enrichment of nucleic acids (such as PCR) and there specific embodiments are well known to the person skilled in the art and well documented in the literature (Sambrook et al. (2001), Molecular Cloning: A laboratory manual, 3^{rd} edition, Cold Spring Harbour Laboratory Press).

It is also possible to use PCR independent accumulation steps based e.g. on affinity chromatography, NAT (nucleic acid amplification testing), ampliphi- or genomiphi-amplification techniques (see product information provided by Amersham Pharmacia), or magnetic microparticles (e.g. Dynabeads™). Samples neither purified, nor accumulated or amplified may also be used in certain circumstances, but require an amplification of the fluorescence signals (signal amplification) in subsequent steps.

For more efficient hybridization of sample molecule and probe molecule it is advantageous if the sample molecule is single-stranded. This can e.g. be achieved if one of the two PCR primers (namely the primer responsible for the synthesis of the strand used for hybridization) carries a chemical modification which protects it from being degraded by a 5′-exonuclease. If this modification is located at the first 5′-nucleotide of the primer, degradation of the respective strand is prevented, while the entire complementary counter-strand, which is not needed for hybridization with the probe molecule, is degraded, i.e. eliminated, by a 5′-exonuclease in 5′-3′-direction starting at the primer position. It is also possible to develop a similar system based on 3′-exonucleases.

Common primer modifications are e.g. 5′-PTO (5′-phosphorothioate nucleotides), carrying phosphorothioate groups instead of phosphate groups at the 5′-terminal nucleotides of the primer. Such modified primers are not degraded by T7 Gen 6 5′-exonucleases or the 5′-exonuclease of the Lambda phage. Other 5′-protective groups or PNA (peptide nucleic acid) primer can be used to protect the hybridizing strand.

A further method for the generation of mainly single-stranded sample molecules, which is not based on modification followed by enzymatic degradation, is the so-called “asymmetric PCR”. In this method, the primer for the required strand is added in excess to the primer for the strand not needed. This results in the preferred synthesis of the strand necessary for hybridization. Hybridization itself is not inhibited by the barely amplified counter strand.

Single-stranded sample molecules may also be obtained via accumulation methods with magnetic particles or affinity chromatography. These substrates contain nucleotides complementary to the target sequence on their surface and extract and accumulate the desired sequences from a denatured, fragmented genomic DNA (e.g. the Kingfischer system of Hybaid).

Of course it is also possible to denature double-stranded nucleic acid molecules, obtained either directly from a sample or via PCR amplification, and to use the mixture of complementary single strands resulting therefrom for hybridization. The sample material needs not necessarily to be present in the form of a separate single strand, but may also be obtained by denaturation and be present together with its complementary strand. This is also the case for asymmetric DNA.

The probe molecules are oligonucleotides or polynucleotides which are able to hybridize with the sample molecules present in the sample due to their nucleotide sequence. These molecules may be DNA or RNA. The sample molecules can be produced according to prior art techniques. They may also be obtained from providers commercially producing oligonucleotides and polynucleotides.

The probe molecules are usually synthetically synthesized single-stranded oligonucleotides, whose last 3′-nucleotide, in cases where primer extension is used as detection method, is situated directly before the polymorphic position in the sequence of the individual to be examined. They are complementary to at least a part of the protected amplified PCR strand, to ensure the compatibility of probe and sample. Furthermore, the extension primers carry a modification at their 5′-end, with which they are attached to the chip surface, which is responsible for the binding to the surface. In a preferred embodiment the modification is a so-called spacer (usually 6 to 24 C-atoms in length; but polyA- or polyT-spacers are known, too), the end of which (opposite to the primer) exhibits an amino modification. This amino group (the so-called linker) reacts with the epoxy coating of the chip when exposed to UV-radiation and therefore chemically and covalently binds the probe molecule to the coating. If the carrier is equipped with a three-dimensional layer, like the CodeLink Bioarray by Amersham Pharmacia, a spacer is not absolutely necessary any more, and the probe may be provided directly with the modification.

As described above, in one embodiment of the invention the probe molecules act as so-called interrogation primer, in prior art terms also referred to as “detection step primer”, which are elongated by primer extension caused by the activity of a polymerase in a template dependent manner. In this case, the primer, i.e. the probe molecule, is complementary to the nucleotide sequence 3′ of the variable nucleotide (SNP) in the corresponding sample molecule. The primer, thereby acting as a starting point for the template dependent elongation by means of a DNA polymerase, is chosen in such a way that it hybridizes with a nucleotide sequence directly adjacent or near the variable nucleotide, which has to be detected within the scope of the SNP analysis. The primer, i.e. the probe molecule, can be chosen to be complementary to either the coding or the non-coding strand of a double-stranded target molecule, depending on which strand should be present in the hybrid of probe molecule and sample molecule.

The choice of the probe molecule, being simultaneously the interrogation primer, is determined by the nature of the nucleotide variation to be analyzed. In one embodiment of the invention the interrogation primer is chosen and produced in such a manner that it is located directly adjacent to the variable nucleotide to be detected after the formation of the hybrid of primer and sample molecule.

In a different embodiment of the invention the interrogation primer is chosen in such a manner that it hybridizes to a probe molecule at a distance of n nucleotide residues from the SNP to be detected. With regard to the number n of nucleotide residues between the 3′-end of the primer and the variable nucleotide, it has only to be considered that no nucleotide residue being identical to the nucleotide to be detected occurs within the n nucleotide residues.

Furthermore, the applied extension primers, being synthesized in a single-stranded form, may exhibit a hybrid nature themselves in the sense that they are only complementary to the sample in their 3′-region, but contain additional sequences in their 5′-region (another approx. 20 bp), which are only complementary to the PCR-primers used (artificial complementary sequences). This allows the creation of a so-called address system, which supports hybridization and enforces the pairing of nucleotides (by the increased number of hydrogen bonds).

Immobilization of oligonucleotides (synthetic probe molecules) on biochips is largely standardized. The cost-efficient alternative is amino-modified probes reacting with epoxysilane-(3-glycidoxypropyltrimethoxysilane-) coated surfaces of fluorescence-free microscope object slides. Additional coatings and corresponding modifications of probes are commercially available. These coatings comprise e.g. aldehyde coatings, aminosilane (3-aminopropytrimethoxysilane) coatings, polylysine coatings or isothiocyanate coatings.

The substrates mentioned preferably react with amino groups at the end of the probe molecules' spacers or of the probe molecules themselves. Another alternative would be the hybrid binding via the biotin/streptavidine system. It is obvious for the person skilled in the art that the functional groups of the coatings and the linker may be interchanged.

Chemical association between the amino groups of the probe molecules and the epoxy groups on the chip is performed by simple UV crosslinking. Alternatively, the chips can be incubated in a humid chamber or heated. At this point it has to be mentioned that surface coating is not restricted to microscope object slides made from glass, but other materials, especially plastics or ceramics may be used for coating. Typically the materials are optically transparent or reflective. The probe molecules are immobilized in the hybridization areas being coated in a plane manner and having a minimum size of 0.5 mm², which are separated from each other by a separating matrix in a preferred embodiment of the invention.

In one application according to the invention, the separating matrix is applied to the chip before or after coating of the chip with the probe molecules, thereby creating separated areas on the chip, in each of which a probe molecule of defined nucleic acid sequence is immobilized in a plane manner. If a separating matrix is used, the matrix can either be pressed onto the carrier in solid form or be applied in a liquid form and then made to harden.

After hybridization and drying of the chip, the separating matrix can be removed, e.g. by stripping it off as a whole with tweezers. This allows a common detection reaction for all hybrids.

According to the invention, the term “hybridization” or “hybridizing” means that two strands of nucleic acid molecules form hydrogen bonds in a sequence dependent manner. Complementary nucleotide sequences can for example hybridize with each other, under suitable conditions known to the person skilled in the art, to form double stranded DNA or RNA or a double stranded hybrid of RNA and DNA. In the context of hybridization see also Sambrook et al., vide supra, Ausubel et al, Current Protocols in Molecular Biology, Green Publishing Associates and Wiley Interscience, New York (1989), or Higgins and Hames, Nucleic Acid Hybridization, A Practical Approach, EAL Press Oxford, Washington D.C. (1985), explicitly incorporated as references.

According to the invention, hybridization is understood to be the formation of double-stranded nucleic acid molecules from complementary single-stranded nucleic acid molecules, wherein complementarity results from the base sequences of the single-stranded molecules. The double-stranded nucleic acid molecules can be DNA-DNA-, DNA-RNA- or RNA-RNA-duplex molecules. Hybridization experiments are usually performed to proof complementarity between different single-stranded nucleic acid molecules.

According to the invention, “annealing” is defined as a process in which two single-stranded nucleic acid molecules attach to each other due to their complementary bases, interact with each other and form double-stranded structures or a double-stranded helix or a duplex. The interaction between the single-stranded nucleic acid molecules is based on hydrogen bonds between complementary base pairs in the individual strands. In this way double-stranded DNA-DNA-helices, DNA-RNA-helices or RNA-RNA-helices can be formed.

Within the scope of the invention, a hybrid is a double-stranded nucleic acid molecule, whose single strands originate from different nucleic acid molecules, and which is generated via the formation of hydrogen bonds between these complementary single strands.

The individual single-stranded sample molecules, resulting from the treatment with the 5′-exonuclease in a preferred embodiment, are preferably brought into contact with the appropriate probe molecules, which are synthesized in a single-stranded form, in spatially defined hybridization fields within the hybridization areas on the chip by means of a microarray robot.

Complementarity is ensured by the positioning of the probe during hybridization at the 3′-end of the genomic single strand (sample).

In one preferred embodiment the hybrids forming are dried by slow evaporation of the reaction liquid. This can take place under normal conditions, via slight heating (25 to 45° C.) or via application of vacuum.

In the next step of a preferred embodiment, the already mentioned primer extension, i.e. the enzymatic extension of the primer which is preferably the probe molecule, takes place in a template dependent manner. The primer extension is generally performed as described by prior art techniques, wherein commercially available enzymes, reagents and kits can be used. A dried hybrid is rehydrated by addition of the extension mixture.

Generally the hybrid of probe and sample is brought into contact with one or more nucleoside triphosphates, including at least one labeled or modified nucleoside triphosphate, and together with a polymerization agent under conditions favorable for the extension of the primer. Either labeled deoxyribonucleoside triphosphates (dNTPs) or labeled chain terminating dideoxyribonucleoside triphosphates (ddNTPs) can be used. The polymerization agent will extend the primer by the nucleoside triphosphate complementary to the variable nucleotide neighboring the primer.

The term “labeled nucleoside triphosphate” includes any nucleoside triphosphate, deoxy- or dideoxynucleoside triphosphate, provided with a detectable marker or modified in such a way that it comprises a group or a residue capable of binding to a detectable marker. Within the scope of the invention, it is not relevant which marker is used for the detection. However, the different markers differ of course with regard to handling, costs and sensitivity. It is important, though, that the detectable marker does not inhibit or cause errors in the incorporation of the labeled nucleotide triphosphate during the polymerization reaction leading to the extension of the primer.

Fluorescence markers for ddNTPs or dNTPs are predominantly cyanine dyes (e.g. Cy3 or Cy5), Renaissance dyes (e.g. ROX or R110) or fluorescent dyes (e.g. FAM or FITC), thus for example cyanine-5-ddATP or Renaissance-110-ddGTP or the corresponding dNTPs. Other fluorescent dyes are developed continuously and are known to the person skilled in the art.

Alternatively, radioactive labels like P³², the biotin/streptavidin system or antigen/antibody systems coupled to enzymes such as horseradish peroxidase may be used.

According to the invention, the detection can be performed with a different number of labeled nucleotides and/or nucleotide analogues. Usually fluorescent dyes are used as markers (see above), and generally either 2 or 4 labeled nucleotides or nucleotide analogues are applied, referred to as “2-color-” and “4-color-approach”, respectively.

Alternatively to this experimental sequence, other primer extension protocols may be applied. One very important method is the allele specific primer extension. The most important difference to the procedure presented above has to be seen in the probes used. The 5′-amino modified probe molecules are designed in such a way that they do not end one nucleotide 3′ before the polymorphic position, but instead, their last 3′-nucleotide is located exactly on the polymorphic position. For this purpose, four probes of identical sequence are required per locus to be analyzed, differing only in the last 3′-nucleotide (the SNP-nucleotide). After application onto four different areas and hybridization with the sample molecule in these four different hybridization areas, the extension reaction elongates only one of the four extension primers, namely the one with the complementary SNP nucleotide at its 3′-terminus. The remaining three extension primers are not extended due to the lack of capability of the last 3′-nucleotide to pair with the sample molecule (extension fails). In order to detect which one of the extension primers has been extended, dNTPs (nucleotides A, C, G and T) labeled with the same fluorochrome are used. The identity of the SNP nucleotide is determined by the detection of the one out of four extension primers showing a fluorescence signal and therefore carrying the correct nucleotide in the last 3′-position.

The detection method described above is not to be mistaken for the allele specific hybridization, in which no extension of a nucleic acid, but only hybridization of probe oligonucleotides and labeled sample in the hybridization solution is performed for detection. In this case the specificity of hybridization is controlled by so-called mismatch oligonucleotides (WO89/10977, Southern et al. (1992), Genomics, 13, 1008-1017). Up to 20 oligonucleotides are required for each locus to be analyzed (due to permutations of the mismatch positions). This detection method is also suitable for the method according to the invention.

The detection of insertions or deletions can also be performed via primer extension analysis. In the case of deletion, it has to be considered that the first base in the deletion needs not to be identical to the first bases behind the deletion. For the detection of insertions, it has to be considered that the first base of the insertion needs not to be identical to the first base behind the insertion.

The term “polymerization agent” refers to any enzyme capable of template dependent extension of nucleic acids. Suitable enzymes include e.g. Sequenase, T7 DNA polymerase, T4 DNA Polymerase, the Klenow fragment of DNA-polymerase from Escherichia coli and other suitable DNA polymerases, reverse transcriptase and polymerases from thermophilic microorganisms like Thermus aquaticus and Thermus thermophilus.

In a preferred embodiment of the invention the polymerization agent is Sequenase™.

The hybrids for the template dependent primer extension are tightly bound in an array format on the chip surface and can be stored in dried form. The performance of multiparallel primer extension, allowing to perform thousands of analyses in e.g. a volume of 50 μl on the chip, is an effective process for economization and cost reduction. If the extension was performed by MTP, by far larger amounts of expensive reagents like Sequenase™ and fluorescent labeled ddNTPs had to be used.

In a preferred embodiment, the extension on the biochip starts when array and extension solution are brought into contact in an evaporation proof chamber on the chip. The reaction mixture containing e.g. 0.2 units of Sequenase™ per μl and 6-8 μM fluorescence labeled ddNTPs in a reaction buffer supplied with the Sequenase™ are incubated together with the array for about 1 h at a temperature optimal for the polymerization agent under these conditions, but not higher than 5° C. below the melting temperature of the complementary region of the probe molecule and the sample molecule, and thus primer extension is performed.

The extension solution may contain all four ddNTPs or analogues, labeled with four different dyes, or it may alternatively contain only two ddNTPs, marked with different dyes, since SNPs often are binary markers themselves, and many applications are only performed to detect which one of the two possible nucleotide states is present. As the labeled ddNTPs are tightly attached to the extension primer (probe molecule) by the polymerization agent and the probe is tightly fixed to the carrier (the chip), the attached ddNTP is also irreversibly bound to the chip.

This allows stringent washing of the chip, e.g. with H₂O, (bidest, 80° C., 10 min) in order to eliminate background noise and to remove non-incorporated ddNTPs. A specific extension of the probe molecules at the sample spot is unambiguously detectable, since the fluorescence wavelength (color) reveals by which ddNTP the primer has been extended at the sample spot. Sample spots typically are of about 80 to 160 μm in diameter when applied automatically, and of 0.5 to 2 mm when applied manually.

In a preferred embodiment the laser-based excitation/detection system analyzing the fluorochromes typically has a resolution of 2 μm and a sensitivity of 0.5 fluorochromes per μmm². In general two (or four) types of fluorochromes are chosen having excitation- and emission-wavelengths as far apart from each other as possible. For example Cy5 is excited at 650 nm and emits at 667 nm (filter 675 nm ±10), R 110 is excited by a 488 nm laser and emits at 525 nm (filter 512 nm ±15). This ensures that the signals of the two dyes do not superpose, since they can be clearly discriminated by appropriate filter systems. The analyzing program compatible with the scanner recognizes the wavelength (color) and intensity of each sample spot and directly transfers the results to a database.

As already described in detail, the method according to the invention allows the simultaneous detection of multiple genomic loci of multiple individuals.

For this purpose, usually several probes, which can be used for the detection of different genomic loci, and which have been immobilized in different hybridization areas, undergo hybridization with the respective complementary sample molecule on the carrier of the chip in distant hybridization fields within the hybridization areas. A maximum of 192 different probes, preferably a maximum of 96 different probes, especially preferably a maximum of 48 different probes, particularly preferably a maximum of different probes, and most preferably a maximum of twelve different probes are immobilized on the carrier.

It is known to the person skilled in the art that the maximum number of probes which can be applied to an array is predetermined by the way of synthesis of the array and the minimum size of the hybridization areas.

According to the invention, the probe molecules are at least 10 to 100 nucleotides in length, preferably at least 15 to 75 nucleotides, especially preferably at least 18 to 50 nucleotides, particularly preferably at least 20 to 40 nucleotides, also particularly preferably from 20 to 35 nucleotides and most preferably at least 20 to 30 nucleotides.

According to the invention, the sample molecules are at least 40 to 500 nucleotides in length, preferably at least 50 to 250 nucleotides, especially preferably at least 50 to 150 nucleotides, particularly preferably at least 50 to 100 nucleotides and most preferably at least 50 to 90 nucleotides.

Preferably, in the method according to the invention, at least 48 different individuals are examined on the carrier, preferably at least 150 different individuals, especially preferably at least 625 different individuals, also especially preferably at least 1250 different individuals, particularly preferably at least 2500 different individuals, and most preferably at least 5000 different individuals.

According to the invention, at least 48 different individuals are examined on the carrier, preferably at least 150 different individuals, especially preferably at least 625 different individuals, also especially preferably at least 1,250 different individuals, particularly preferably at least 2,500 different individuals, and most preferably at least 5,000 different individuals are examined in the method in parallel with respect to a maximum of 192 different loci, preferably a maximum of 96 different loci, especially preferably a maximum of 48 different loci, particularly preferably a maximum of 24 different loci and most preferably a maximum of twelve different loci.

According to the invention different individuals of different organisms can be examined with regard to genetic polymorphisms like SNPs. These include e.g. microorganisms, preferably E. coli and/or human pathogenic microorganisms, fungi, preferably Saccharomyces cerevisiae, Schizosaccharomyces pombe and/or plant pathogenic fungi, rats, cattle, mice, humans or plants.

In the case of plants, preferably individuals of monocotyledonous or dicotyledonous economic plants, ornamental plants, food plants or fodder plants are characterized by the method according to the invention. Examples for monocotyledonous plants are plants of the genera Avena (oats), Triticum (wheat), Secale (rye), Hordeum (barley), Oryza (rice), Panicum, Pennisetum, Setaria, Sorghum (millet), Zea (corn), and suchlike. Dicotyledonous economic plants comprise among others cotton, leguminous plants like pulse and especially Alfalfa, soy bean, rape, tomato, sugar beet, potato, ornamental plants and trees. Further economic plants can be fruits (especially apple, pear, cherry, grape, citrus, pineapple and banana), oil palm trees, tea-, cocoa- and coffee-shrub, tobacco, sisal and from the realm of medical plants Rauvolfia and Digitalis. Especially preferred are the crops wheat, rye, oats, barley, rice, corn and millet, sugar beet, rape, soy, tomato, potato and tobacco. Further economic plants can be taken from the U.S. patent U.S. Pat. No. 6,137,030. According to the invention, most preferred are Arabidopsis as model system for dicotyledonous plants and rice as model system for monocotyledonous plants.

One or more human individuals can be examined e.g. with regard to one or more polymorphisms inter alia in the genes for 5-lipoxygenase or Cytochrome P450, which result in a modified metabolism of active agents. Thus, the active agent therapy could be adjusted individually to the genotype of the respective individual.

Of course, the characteristic feature of the method according to the invention, i.e. the spatially defined hybridization of one sample molecule with one probe molecule in a hybridization field, is also applicable for nucleic acid analogues like PNAs. Sample and probe may also be of totally different nature, e.g. proteins or peptides for antibody screening or carbohydrates like sugar chains at glycoproteins for the screening of low molecular ligands (antigens) of all classes of substances.

The method according to the invention can therefore be applied to all detection methods in which the specific interaction between two molecules (probe and sample) exhibiting mutually complementary binding properties is detected via the formation of a hybrid and in parallel.

Insofar the method according to the invention is not restricted to the detection of SNPs, but can also be used for the parallel detection of multiple species in one sample, e.g. via rDNA-hybridization. Also, the genetic activity of a cell and the expression of certain genes can be detected by the method according to the invention.

It is known to the person skilled in the art, that the detection of the hybrids on the chip can also be performed by a number of additional detection methods which have not been mentioned so far. For the rDNA-based detection of microorganisms the sample molecules may e.g. be labeled by the use of appropriately labeled primers already during amplification, e.g. with the already mentioned fluorophors. The detection method consists of determining whether the labeled hybrids can be detected on the microarray. The same detection method can be applied for the analysis of genetic activity, if during PCR amplification of the cDNA (see above) appropriately labeled primers are applied. Further detection methods are known to the person skilled in the art.

A method according to the invention can usually comprise the following steps:

In the following, an example is first presented, showing in an exemplary manner how multiple SNPs of multiple individuals can be detected simultaneously on a chip by a method according to the invention. It is common knowledge to the person skilled in the art how the method can be adapted to his requirements via optimization and variation of the individual experimental steps. The examples are not intended to be restrictive.

First, singular SNPs are identified via bioinformatics and public sequence databases. The different genome projects provide millions of sequences of expressed genes (cDNAs), but also total genomes. Additionally, databases are available (e.g. at the NCBI, http://www.ncbi.nim.nih.gov) indicating explicitly the positions of SNPs in the genomes of different organisms. Starting from these SNP databases or by comparison of own sequence data with the corresponding reference sequences in public databases SNPs can be identified.

In a second step, the primers for PCR are deduced, which amplify the genomic fragment that carries the polymorphism to be detected. A part of one of the two primers has to have the same sequence as a part of the future extension primer (the probe), in order to ensure hybridization of the probe with the target with 100% certitude. The primer used for amplification of the strand required for hybridization can carry a modification which protects from degradation by a 5′-exonuclease (e.g. a phosphorothioate modification). Primers may be designed e.g. with the Primer3 software (Whitehead Institute, MIT, Steve Rozen, Helen J. Skaletsky, 1996 and 1997; available at http://www.genome.wi.mit.edu/genome_software/other/primer3.html).

Optionally, comparative sequencing of the amplified fragments in different genetic backgrounds can be performed for verification, quality control and the choice of suitable markers.

In the next step, 5′-amino modified probe molecules are also designed using the Primer3 software and synthesized. Orders are placed at well-known manufacturers like Metabion, Martinsried, Germany. The probes are chosen in such a way, that they terminate at their 3′-end exactly one nucleotide before the polymorphic position of the polymorphism to be examined. The single-stranded probe molecules are used for plane coating of the biochips in areas. This coating is performed by companies specialized in coating of surfaces with DNA molecules, such as RoboScreen, Leipzig, Germany. Alternatively, coating of epoxy-modified biochip templates (e.g. Epoxy Slides from Quantifoil, Jena) can be performed by the experimenter himself.

Either before or after coating with the probe molecules, a separating matrix, e.g. made from silicone rubber, may be applied, providing additional separation of hybridization areas against each other.

Using the already mentioned PCR primer, the different genomic loci of the different individuals to be examined are then amplified via PCR. The annealing temperature of the primers is between 50 and 68° C., preferably between 55 and 60° C. The size of the amplified fragments is between 40 to 500 base pairs, preferably between 50 and 250 base pairs, especially preferably between 50 and 150 base pairs, particularly between 50 and 100 base pairs and most preferably between 50 and 90 base pairs.

Typical reaction conditions comprise:
10 ng/μl genom. DNA template
0.2 mM dNTPs
1.5 mM MgCl₂
10× reaction buffer
5 μM each of the forward and the reverse primer
0.02 U/μl Taq polymerase

Polymerase and reaction buffer are typically obtained from Qiagen, Germany.

The temperature protocol for the PCR is usually the following:

| | | | |
|---|---|---|---|
| 1 cycle: | 5 min | at | 94° C. |
| 35 cycles: | 20 sec | at | 94° C. |
| | 30 sec | at | annealing temperature (50 to 68° C.) |
| | 25 sec | at | 72° C. |
| 1 cycle | 2 min | at | 72° C. |

Subsequently, the unprotected amplified strand, showing the same sequence as the probe molecule, is digested by an exonuclease with high selectivity for the PTO modification, e.g. T7 Gen 6 5′-exonuclease, according to the manufacturer's instructions (Amersham-Pharmacia Biotech).

After inactivation of the 5′-exonuclease (e.g. incubation for 5 min. at 90° C.), the single-stranded sample is brought into contact with its complementary probe oligonucleotide in a hybridization field of the hybridization area, using a microarray robot (e.g. Microgrid® II 600, Biorobotics) or pipette robot (e.g. Hamilton Microlab® Star), or a manual pipette, and thus hybridization is initiated. The positions of the probe and the examined individuals are known.

In a preferred embodiment the forming hybrids are dried by evaporating the liquid.

After hybridization the separating matrix, if present, can be removed by stripping it without residue from the carrier with pincers. This results in one single large reaction filed, on which the detection reaction is performed. If different extension mixtures are to be used in the different areas, the separation matrix may remain on the object slide. If the same extension mixture should be used in more than one area, but not in all of them, more than one separation matrix may be applied, wherein for certain separation matrices the extension is removed and others remain on the carrier. This allows an analysis as cost-efficient as possible.

In the next step, the extension reaction is performed on the chip, using Sequenase™ and differently fluorescence labeled ddNTPs in Sequenase™ buffer at 50-70° C., but in any case below the melting temperature of the hybrid. For this purpose, the chip with the reaction mixture is enclosed in an evaporation-proof manner. This is provided either by the remaining separation matrix, or, if no separating matrix has been used or the separating matrix has already been removed after hybridization, by means of a silicone rubber sealing (e.g. Casil 401T) which encloses the object slide and seals the object slide against a lid (second object slide or coverslip).

SNP detection is performed in this case by the extension of the hybrid molecule on the chip surface by a fluorescence labeled nucleotide (so-called “single base extension”), the probe oligonucleotide thereby acting as primer and the hybridized single-stranded sample as template. Since the nucleotides carry different fluorescence markers and cannot be further extended during the extension reaction, only this nucleotide is incorporated which is complementary to the respective SNP.

After performance of the extension reaction the chip is washed once with hot distilled water, which may contain up to 0.1-2% SDS, and once with 1× SSC plus 0.1% SDS, then rinsed with distilled water and dried in a stream of nitrogen. Then the chip is inserted into a laser scanner (e.g. GSI LS IV, GSI Lumonics or Typhoon, Amersham Pharmacia) or a different scanner (e.g. StormReader, Molecular Dynamics or ImageScanner, Amersham Pharmacia) which detects within a few seconds the color (emitted wavelength) of the extended probe molecule, and recognizes the identity of the SNP at the respective sample spot on the chip on the basis of the measured wavelength. In this regard, the physical properties of the dyes have to be considered in the choice of laser/filter systems according to the manufacturer's instructions. (e.g. GSI Lumonics). The results are directly recorded as fluorescence intensity values (e.g. 65536 shades of gray at the wavelengths of the fluorescence labeled ddNTPs) in the database.

In the following the invention is explained by means of a specific example. This is not intended to be understood as restrictive.

### EXAMPLE

### Example 1

### Detection of 18 Loci in 4 Cultivars

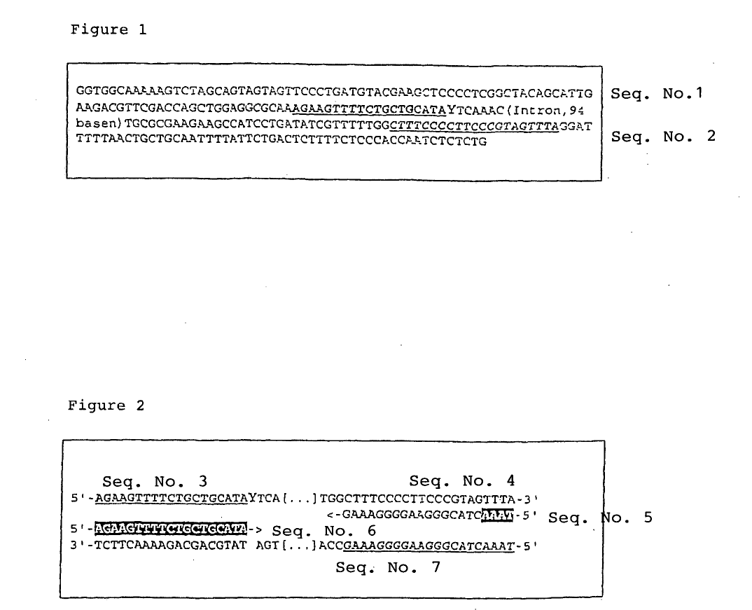

The detection of 48 loci in 4 cultivars of barley is described in the following. As an example, FIGS. 1 to 3 illustrate the positions of the PCR primers with respect to the sequence of an examined locus. In this example an SNP is investigated with regard to thymine (T) and cytosine (C).

First, the sequence information of 48 single nucleotide polymorphisms (SNPs) of Hordeum vulgare L was determined by means of bioinformatics in EST- and genomic sequence databases with NCBI (http://www.ncbi.nlm.nih.gov). The exemplary locus has the accession code: gi9410596.

Then, the Primer3 software (Whitehead Institute, Mass. Institute of Technology (MIT) Steve Rozen, Helen J. Skaletsky, 1996, 1997) was used to design genomic PCR primers for the amplification of the sample molecule. Compilation of the source code using SuSe Linux, Kernel 2.4. was performed according to the instructions of the programming engineer in the documentation supplied with the source code and from the web site (http://www.genome.wi.mit.edu/genome_software/other/primer3.html).

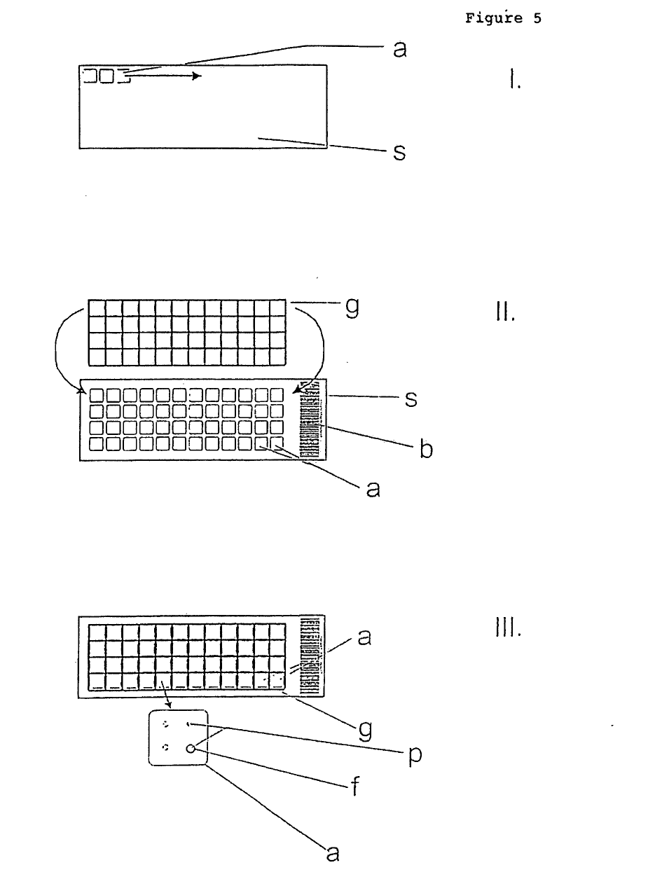

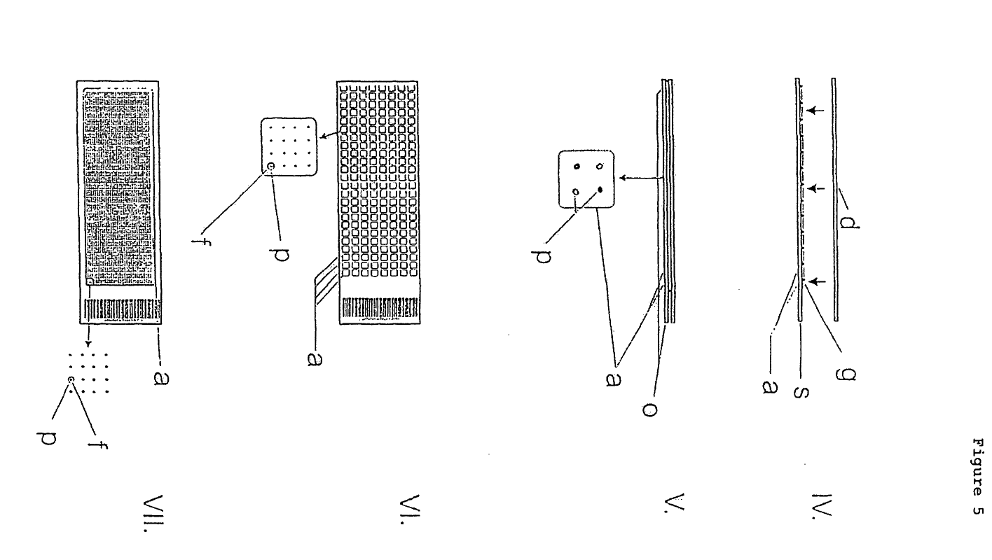

The genomic primers were designed in such a way that they amplify genomic fragments which carry the SNPs to be examined (figure (fig.) 1, Y on gray background). One of the two primers carries phosphothioate bond modifications at the 5′-end, and the other, non-modified primer, corresponds in sequence to the later extension primer (probe, FIGS. 3 and 4, oligonucleotide in box), to ensure the error-free hybridization of the probe, starting with the 3′-end, with the single-stranded PCR fragment after the amplification. The reverse primer (PTO-primer), carrying the hybridizing half-strand, requires a modification to protect it from degradation by a 5′-exonuclease.

Then, a comparative sequencing (ABI Sequenzer 3700, Rhodamin Sequencing Kit, Applied Biosystems, USA) of the amplified PCR fragments with one of the two PCR primers in different genetic backgrounds was performed for verification, quality control and choice of suitable markers.

Subsequently, 5′-aminomodified probe molecules were designed, ending 3′ exactly one nucleotide before the polymorphic position (software Primer3, see above, FIGS. 3 and 4, oligonucleotide in box). Synthesis of the probes was ordered at Metabion GmbH, Germany. The probe molecules carried an NH₂-group as functional linker group, which was attached to the probe via a C₁₂ spacer. These probe molecules were used for the production of 48 oligonucleotide areas having a surface of 16 mm² and a distance of 1 mm. A solution of at least 500 fmol/μl of the C₁₂-aminomodified oligonucleotide was used, in order to ensure that even a binding frequency of one out of 1000 oligonucleotide molecules is sufficient to pass the detection limit of the fluorescence scanning device (GSI Scanner IV, Packard Bioscience). This solution was applied to the slide (s) with 50% QMTTM buffer (Quantifoil, Jena) at spots of about 150 μm in diameter and at a distance of 100 to 150 μm using a microarray robot, allowing the droplets to combine and leading to a continuous coating in square areas (a) (FIG. 5/I). A density of 0.5 to 5 probe molecules per μm² was achieved.

Subsequently, the binding reaction was carried out:
5 min at 4° C.
let humidify in a clean laboratory (absence of soluble, pH-affecting reactive gases) at room temperature (25° C.) and at least 60% humidity (humid, but not wet)
then UV crosslinking applying 2×300 J/cm² for irreversible binding of the hybrid molecules to the chip surface.

This results in the reaction of the epoxy group of the chip to form covalent bonds with the 5′-amino groups at the C₁₂-spacer of the probe molecules which are thereby fixed to the chip surface.

After the binding reaction, the grid-like separating matrix (g) (made of the transparent and thermostable elastomer CASIL 401 T, obtained from Incasil GmbH, Ludwigsburg, Germany, made to measure) was applied, so that the hybridization areas (a) were additionally separated from each other (FIG. 5/II). For its identification, the slide (s) was equipped with a bar code (b), which can be detected by a reader.

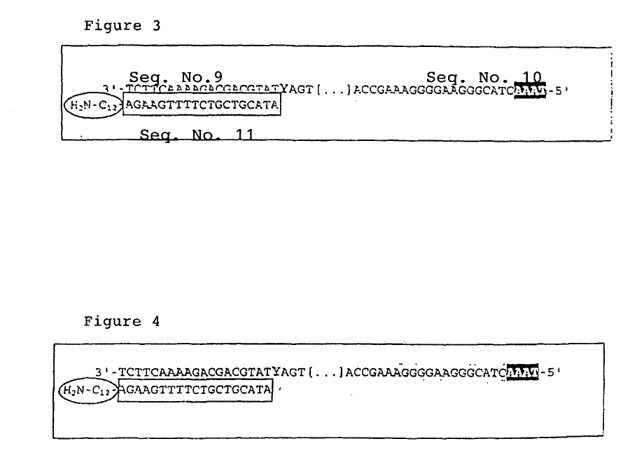

Subsequently the 48 PCR fragments from the genomic DNA of the 4 cultivars were amplified. An example for genomic primers at the locus is given in FIG. 2.

In the example, the annealing temperature was 51° C. The amplified fragment size was 174 base pairs. The PCR was performed according to the instructions of the polymerase manufacturer (Qiagen GmbH, Germany) following the protocol given below:

| | | |
|---|---|---|
| 10 | ng/μl | genomic DNA |
| 0.2 | mM | dNTPs |
| 1.5 | mM | MgCl₂ |
| 10× | | Taq reaction buffer (according to the manufacturer's |
| | | instructions) |
| 5 | μM each | forward and reverse PCR primer |
| 0.02 | Units/μl | Taq polymerase |

The following temperature protocol was used:

| | | | |
|---|---|---|---|
| 35 cycles: | 20 sec | at | 94° C. |
| | 30 sec | at | 51° C. |
| | 25 sec | at | 72° C. |

After that, the amplified PCR fragments were precipitated by addition of 2 volumes (vol) of pure ethanol and 1/10 vol of 3 M sodium acetate pH 4.5 at 4° C., and centrifuged at 14,000 rpm and 4° C. for 45 min. Afterwards the precipitate was washed twice by addition of 70% ethanol and again centrifuged at 14,000 rpm and 4° C. for 15 min. After drying, the pellet was dissolved in 6 μl of distilled water.

The (counter) strand in the fragments, which was not provided with 5′-PTO bonds and was therefore unprotected, was degraded thereafter by means of the 5′-exonuclease T7 Gen 6 (Amersham Pharmacia-Biotech) which is selective in respect of PTO, while protecting the DNA strand which is complementary to the probe molecule and relevant for the analysis.

This is the protocol used in accordance with the manufacturer's instructions:

| | |
|---|---|
| 2 μl | precipitated PCR product |
| 0.1 μl | exonuclease |
| 1.9 μl | dist. water |
| 1.0 μl | 5× exonuclease buffer (see manufacturer's instructions) |

After incubation for 60 min at 37° C. the 5′-exonuclease was inactivated by heating for 20 min at 85° C. The solution containing the single-stranded sample molecule in the exonuclease buffer was diluted with polyethylene glycol (having a polymerization degree of 3,000-5,000 subunits) to a final concentration of 0.2%.

Afterwards, 2 μl of the single strand sample (p), free of counter strand, are pipetted onto a hybridization field (f) in the hybridization area (a) separated by the separating matrix (g) by means of a microarray robot (Microgrid® II 600, Biorobotics). The hybridization area contains the oligonucleotide which comprises the complementary region adjacent to the SNP (FIG. 5/III). The droplet was dried by slow evaporation (5 to 15 min) of the liquid, thereby forming the hydrogen bonds between probe molecule and sample molecule. In order to slow down the evaporation, the system was kept above a water bath, causing a humidity of 60 to 70%. Hybridization of the probe molecule and the sample molecule occurred during drying (see FIG. 3). During this reaction the other hybridization fields of the hybridization area (three in this example) were charged with the sample molecules at a distance of 0.3 mm using a microarray robot (see above).

Afterwards 30 μl of the extension reaction mixture of the following composition:

| | | |
|---|---|---|
| 6 | μl | 5× Sequenase buffer (Amersham) |
| 2.4 | μl | 1 μM Cy5-ddATP (NEN/PerkinElmer) |
| 2.4 | μl | 1 μM R110-ddGTP (PerkinElmer) |
| 6.1 | U | Sequenase ™ |
| ad 30 | μl | dist. Water |

was applied to the corresponding hybridization area (a) of the array. The extension reaction was carried out at 50° C. for 1 h in the evaporation-proof chamber construction of the Thermocycler in situ PCR system 1.000 (Perkin Elmer in situ PCR System 1.000 plus accessories). The evaporation-proof chamber construction is formed as the remaining separating matrix (g) seals the object slide (s) against a second object slide acting as lid (d) (FIG. 5/IV). The lid was applied from one side onto the separating matrix and the extension mixture, avoiding air bubbles, and fixed with a clamp (AmpliCover Clip, PerkinElmer). FIG. 4 shows the extension reaction for the described SNP Y.

The chip was then cooled on ice for 5 sec and washed with hot dist. water at 80° C. and with 1× SSC with 0.1% SDS at 60° C. immediately after opening the clamp (Sambrook et a, 2001, vide supra). After brief rinsing with dist. water the chip was dried in a stream of nitrogen.

The chip was analyzed using the laser scanner GSI LS IV (GSI Lumonics). For this purpose the sample spots containing the hence fluorescent labeled, elongated extension primers were excited with monochromatic light at 488 and 650 nm, and the emitted light was detected at 525 nm and 667 nm and recorded. The physical properties of the dyes were taken into account for the choice of the laser/filter systems and the laser energies used, according to the advice of the manufacturer (manual supplied by Genomic Solutions, USA). Thereby, the color (emitted wavelength of the fluorescent labeled nucleotide) of the elongated probe molecule and therefore the identity of the SNPs at the respective sample spots on the chip were determined.

The results (base status in the respective SNP position) were stored as fluorescence intensity values directly in the database. GT Scan Software and GSI Analyzer (as well as according documentation) were used.

What is claimed is:

1. A method for the detection of nucleic acids, comprising the following steps in the order given:
a) plane arrangement and immobilization of one or more single-stranded nucleic acid molecules (probe molecules) in hybridization areas having a minimum size of 0.5 mm² on a carrier,
b) mixing and hybridization of a specific probe molecule in a spatially defined hybridization field within the hybridization area with a second single-stranded nucleic acid molecule (sample molecule), whose sequence is at least partly complementary to that of the probe molecule, therefore allowing the hybridization of the two nucleic acid molecules at least over the range of the complementary sequence, wherein sample molecules of the same locus, but of different origin, can hybridize with the same probe molecule in different hybridization fields within one hybridization area,
c) detection of the hybridization with a suitable detection method.

2. The method of claim 1, wherein the hybrids forming in a hybridization field are dried, before probe molecule and sample molecule are brought into contact with each other in the neighboring hybridization field.

3. The method of claim 1, wherein the hybridization areas are separated from each other by a removable separation matrix.

4. The method of claim 1, wherein the surface of the hybridization areas is at least 0.5 mm², preferably at least 4 mm², especially preferably at least 16 mm² and most preferably at least 256 mm².

5. The method of claim 1, wherein the distance between the hybridization fields is at least 1.5 to 2 mm in the case of manual application of the sample molecule, and at least 0.1 to 1 mm in the case of automatic application.

6. The method of claim 3, wherein the separating matrix consists of a tear resistant, chemically inert material which seals against the carrier.

7. The method of claim 6, wherein the material for the separating matrix is silicone rubber.

8. The method of claim 1, wherein the sample molecule contains a single nucleotide polymorphism (SNP) to be detected.

9. The method of claim 8, wherein probe molecules representing different loci and sample molecules from different individuals are used for parallel analysis.

10. The method of claim 8, wherein the detection of the SNP is performed by means of the analysis of the hybridization reaction via template dependent extension of the probe molecule, acting as a primer within the hybrid, and via incorporation of labeled nucleotides or nucleotide analogues.

11. The method of claim 10, wherein the detection is performed by means of template dependent enzymatic extension of the probe molecule, acting as a primer within the hybrid, and via incorporation of a labeled nucleotide or nucleotide analogue leading to chain termination.

12. The method of claim 10, wherein the nucleotide analogues are ddNTPs and N stands for A, T, C, G.

13. The method of claim 10, wherein the molecules used for labeling the labeled nucleotide comprise cyanine dyes, preferably Cy3 and/or Cy5, Renaissance dyes, preferably ROX and/or R110, and/or fluorescent dyes, preferably FAM and/or FITC.

14. The method of claim 1, wherein the single-stranded sample molecules are obtained by means of PCR amplification and selective enzymatic digestion of one strand, by means of asymmetric PCR and/or by means of affinity purification of one of the amplified strands.

15. The method o f claim 1, wherein the single-stranded probe molecules are obtained by chemical synthesis.

16. A carrier for the detection of nucleotide polymorphisms, on which probe molecules are arranged and immobilized in a plane manner in hybridization areas having a minimum size of 0.5 mm², and which contains a removable separation matrix made of chemically inert material by which the hybridization areas are separated from each other.

17. A kit for the detection of nucleotide polymorphisms, comprising a carrier according to claim 16.

18. A kit for the detection of nucleotide polymorphisms, comprising a carrier according to claim 16, one or more primers for the PCR amplifications according to claim 14, and reagents for the generation of single strands from the PCR products according to claim 14.

19. A kit for the detection of nucleotide polymorphisms, comprising a carrier according to claim 16, one or more primers for the PCR amplifications according to claim 14, reagents for the generation of single strands from the PCR products according to claim 14 and reagents for the template dependent extension according to claim 10.
